(19) United States
(12) Patent Application Publication
(10) Pub. No.: US 2008/0312694 A1
(43) Pub. Date: Dec. 18, 2008

(54) DYNAMIC STABILIZATION ROD FOR SPINAL IMPLANTS AND METHODS FOR MANUFACTURING THE SAME

(76) Inventors: Marc M. Peterman, Austin, TX (US); Derrick William Johns, Austin, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/763,969

(22) Filed: Jun. 15, 2007

(51) Int. Cl. A61B 17/70; A61B 19/00

(52) U.S. Cl. 606/257; 128/898; 606/254

(57) ABSTRACT

Embodiments of the disclosure provide a new and improved dynamic stabilization rod for spinal implants and methods of making the same The dynamic stabilization rod has a hollow cylindrical body and an opening extending spirally around a longitudinal axis of the cylindrical body. The cylindrical body, including the opening, may be filled and/or coated in whole or in part with polycarbonate urethane to prevent over extension and reduce wear. The opening may be machined or otherwise cut to a shape corresponding to a dog bone or puzzle. Portion(s) of the cylindrical body can be left rigid and uncut for integration with other spinal device(s) to facilitate fusion or segmental stability of the spine.

### TECHNICAL FIELD

This disclosure relates generally to spinal implants, and more particularly to dynamic stabilization rods for spinal implants and methods for manufacturing the same.

### BACKGROUND

Modern spine surgery often involves spinal fixation through the use of spinal implants or fixation systems to correct or treat various spine disorders or to support the spine. Spinal implants may help, for example, to stabilize the spine, correct deformities of the spine, facilitate fusion, or treat spinal fractures. A spinal fixation system typically includes corrective spinal instrumentation that is attached to selected vertebra of the spine by screws, hooks, and clamps. The corrective spinal instrumentation may include spinal rods or plates that are generally parallel to the patient's back. The corrective spinal instrumentation may also include transverse connecting rods that extend between neighboring spinal rods. Spinal fixation systems are used to correct problems in the cervical, thoracic, and lumbar portions of the spine, and are often installed posterior to the spine on opposite sides of the spinous process and adjacent to the transverse process.

Various types of screws, hooks, and clamps have been used for attaching corrective spinal instrumentation to selected portions of a patient's spine. Examples of pedicle screws and other types of attachments are illustrated in U.S. Pat. Nos. 4,763,644; 4,805,602; 4,887,596; 4,950,269; and 5,129,388. Each of these patents is incorporated by reference as if fully set forth herein.

Often, spinal fixation may include rigid (i.e., in a fusion procedure) support for the affected regions of the spine. Such systems limit movement in the affected regions in virtually all directions (for example, in a fused region). More recently, so called “dynamic” systems have been introduced wherein the implants allow at least some movement of the affected regions in at least some directions, i.e. flexion, extension, lateral, or torsional. While at least some known dynamic spinal implant systems may work for their intended purpose, there is always room for improvement.

### SUMMARY

This disclosure provides embodiments of a new and improved dynamic stabilization rod for spinal implants and methods of making the same. For example, in accordance with one feature of the disclosure, a dynamic stabilization rod having a cylindrical body with a cannulated or otherwise hollow interior is provided for use in an implant system that supports a spine.

In accordance with one feature of the disclosure, a dynamic stabilization rod has a hollow cylindrical body and an opening extending spirally around a longitudinal axis of the cylindrical body. The opening may be machined, etched, or otherwise cut to a shape. According to one feature of the disclosure, the shape has a non-linear path. In one embodiment, the opening has a linear path. In one embodiment, the shape has an interlocking pattern. In one embodiment, the shape resembles a dog bone. In one embodiment, the shape resembles a puzzle.

In accordance with one feature of the disclosure, a portion or portions of the cylindrical body can be left rigid and uncut for integration with other spinal device(s) such as bone fasteners to facilitate fusion or segmental stability of the spine Examples of bone fasteners include pedicle screws, hooks, clamps, wires, interspinous fixation devices, injectable nuclei, etc.. In one embodiment, the cylindrical body has a mid section and the opening extends longitudinally about the mid section. In one embodiment, the opening extends from one end of the cylindrical body to the other.

In accordance with one feature of the disclosure, a dynamic stabilization rod may be filled and/or coated in whole or in part with a biomaterial such as a polymer to prevent over extension and reduce wear. In one embodiment, the polymer is polycarbonate urethane. In one embodiment, the opening of the dynamic stabilization rod is at least partially filled with the polymer to enhance rigidity of its cylindrical body.

In accordance with one feature of the disclosure, the cylindrical body and the opening of a dynamic stabilization rod may be filled in whole or in part with the same biomaterial or different biomaterials. In one embodiment, the cylindrical body and opening of a dynamic stabilization rod may be filled in whole or in part with polycarbonate urethane.

In accordance with one feature of the disclosure, a dynamic stabilization rod may be coated in whole or in part with a biomaterial such as a polymer. In one embodiment, the polymer is polycarbonate urethane.

In accordance with one feature of the disclosure, a dynamic stabilization rod in use extends along the length of the spine and connects a set of bone fasteners affixed to the spine. In one embodiment, the dynamic stabilization rod and the set of bone fasteners are part of a spinal stabilization system. In one embodiment, the set of bone fasteners are pedicle screws.

In accordance with one feature of the disclosure, a spinal stabilization system is provided for supporting a spine. The system includes first and second dynamic spinal rods to be fixed on laterally opposite sides of a spine.

According to one feature of the disclosure, a dynamic stabilization rod can be made by a method comprising the steps of forming a cylindrical body from a first biomaterial; removing the first biomaterial from inside of the cylindrical body along a longitudinal axis of the cylindrical body to form a cannulated interior, machining an opening about the longitudinal axis of the cylindrical body, at least partially filing the opening with a second biomaterial to enhance rigidity of the dynamic stabilization rod, wherein the second biomaterial is a polymer, and coating the cylindrical body in whole or in part with the polymer.

In one embodiment, the steps of the aforementioned method of making a dynamic stabilization rod are performed in order. In one embodiment, the steps of the aforementioned method of making a dynamic stabilization rod are performed in no particular order.

In one embodiment, the method of making a dynamic stabilization rod further comprises filling the cannulated interior in whole or in part with the second biomaterial. In one embodiment, the second biomaterial is polycarbonate urethane.

In one embodiment, the method of making a dynamic stabilization rod further comprises rotating the cylindrical body around the longitudinal axis, moving the cylindrical body in an axial direction, and following a predetermined non-linear path, continuously or intermittently cutting away the first biomaterial from the cylindrical body. In one embodiment, the predetermined non-linear path corresponds to a recurring pattern of a dog bone or puzzle.

In one embodiment, the aforementioned cutting is performed utilizing a computer-controlled technique. In one embodiment, the computer-controlled cutting technique includes rotating and moving the cylindrical body utilizing a computer-controlled mechanical device and directing one or more lasers to follow the predetermined non-linear path and continuously or intermittently cut away the first biomaterial from the cylindrical body using the one or more lasers

Other features, advantages, and objects of the disclosure will be better appreciated and understood when considered in conjunction with the following description and the accompanying drawings.

### BRIEF DESCRIPTION OF THE DRAWINGS

A more complete understanding of the present disclosure and the advantages thereof may be acquired by referring to the following description, taken in conjunction with the accompanying drawings in which like reference numbers indicate like features and wherein:

FIG. 1 depicts a simplified diagrammatic representation of a top view showing a spinal implant system in use and including a pair of dynamic stabilization rods according to one embodiment of the disclosure;

FIG. 2 depicts a schematic representation of a dynamic stabilization rod and an exploded view showing a detailed portion thereof according to one embodiment of the disclosure.

FIG. 3 depicts a schematic representation of a dynamic stabilization rod and exploded views showing detailed portions thereof according to one embodiment of the disclosure;

FIGS. 4-5 depict schematic representations of dynamic stabilization rods with varying features according to some embodiments of the disclosure; and

FIGS. 6A-6K depict schematic representations of various patterns for implementing embodiments of dynamic stabilization rods disclosed herein;

FIGS. 7A-7C depict schematic representations of side views each showing a portion of a spiral opening of a dynamic stabilization rod having a distinct pitch according to some embodiments of the disclosure;

FIG. 8 depicts a schematic representation of a side view of a portion of a spiral opening of a dynamic stabilization rod in a normal state;

FIGS. 9-10 depict schematic representations of side views of the portion of the spiral opening of the dynamic stabilization rod of FIG. 8 under rotational forces;

FIG. 11 depicts a schematic representation of a dynamic stabilization rod with more than one spiral opening according to one embodiment of the disclosure; and

FIG. 12 depicts a schematic representation of a top view showing a spinal implant system in use and including a pair of dynamic stabilization rods connected via a cross-link according to one embodiment of the disclosure.

### DETAILED DESCRIPTION

The disclosure and the various features and advantageous details thereof are explained more fully with reference to the non-limiting embodiments detailed in the following description Descriptions of well known starting materials, manufacturing techniques, components and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, and additions within the scope of the underlying inventive concept(s) will become apparent to those skilled in the art after reading this disclosure. Skilled artisans can also appreciate that the drawings disclosed herein are not necessarily drawn to scale.

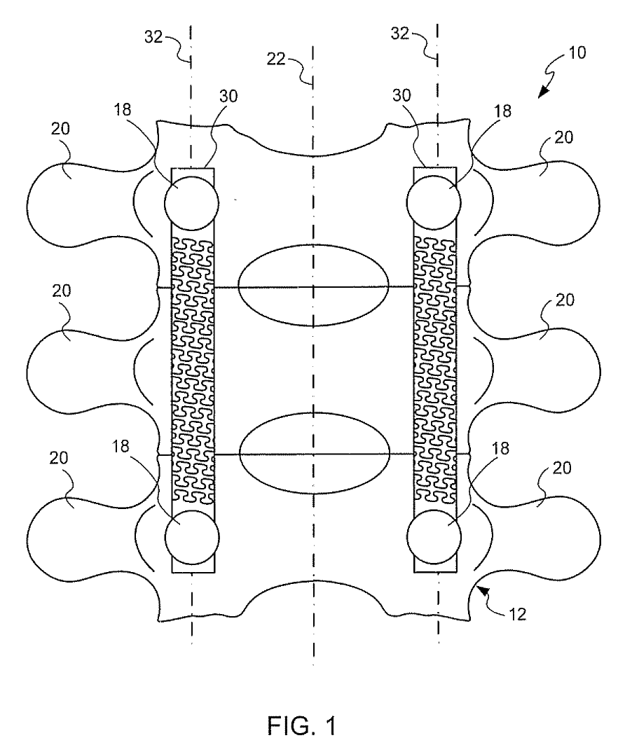

With reference to FIG. 1, a fixation or implant system 10 for supporting a spinal column 12 includes a pair of dynamic stabilization rods 30. In this example, only one pair of dynamic stabilization rods 30 is shown. However, one skilled in the art can appreciate that more than two dynamic stabilization rods 30 may be utilized in a spinal procedure. As illustrated in FIG. 1, dynamic stabilization rods 30 can be fixed laterally on opposite sides of the spine 12 to selected vertebra 20 of the spine 12, utilizing anchor systems 18. As an example, anchor systems 18 may comprise bone fasteners such as pedicle screws, hooks, claims, wires, etc. Components of the system 10 are made from biocompatible material(s). Examples of biocompatible materials include titanium, stainless steel, and any suitable metallic, ceramic, polymeric, and composite materials.

In embodiments disclosed herein, the term “dynamic” refers to the flexing capability of a spinal rod. The flexing capability is configured to provide a bending stiffness or a spring rate that is non-linear with respect to the bending displacement of the rod. This is intended to more closely mimic the ligaments in a normal stable spine which are non-linear in nature. The non-linear bending stiffness of the dynamic stabilization rods disclosed herein is intended to allow the limited initial range of spinal motion and to restrict or prevent spinal motion outside of the limited initial range. In one embodiment, the bending stiffness is produced by configuring the rod to provide a first bending stiffness that allows the initial range of spinal bending and a second bending stiffness that restricts spinal bending beyond the initial range of spinal motion. One way to achieve both the first bending stiffness and the second bending stiffness is to configure the opening of the rod to have a lower bending moment of inertia I (sometimes referred to as the second moment of inertia or the area moment of inertia) through the initial range of spinal motion and a higher bending moment of inertia beyond the initial range of spinal motion.

Thus, with dynamic stabilization rods 30, the system 10 can allow a limited range of spinal bending, including flexion/extension motion. While the range of bending may vary from patient to patient, the system 10 can allow sufficient spinal bending to assist the adequate supply of nutrients to the disc in the supported portion of the spine 12. Movement beyond the initial range of motion is restricted by the system 10 so as not to defeat the main purpose of the fixation system 10.

The system 10 is installed posterior to the spine 12, typically with the rods 30 extending parallel to the longitudinal axis 22 of the spine 12 lying in the mid-sagittal plane. According to one feature of the disclosure, the system 10 can include additional rods positioned further superior or inferior along the spine 12, with the additional rods being dynamic stabilization rods such as the rods 30, or other types of non-dynamic or rigid rods. It should be understood that the system 10 may also include suitable transverse rods or cross-link devices that help protect the supported portion of the spine 12 against torsional forces or movement. Some possible examples of suitable cross-link devices are shown in co-pending U.S. patent application Ser. No. 11/234,706, filed on Nov. 23, 2005 and naming Robert J. Jones and Charles R. Forton as inventors (the contents of this application are incorporated fully herein by reference). Other known cross-link devices or transverse rods may also be employed. According to one feature of the disclosure, the dynamic stabilization rods 30 are configured to possess sufficient column strengthen and rigidity to protect the supported portion of the spine 12 against lateral forces or movement.

According to one feature of the disclosure, each of the dynamic stabilization rods 30 extends along a longitudinal axis 32 in an un-deformed state. In embodiments of the disclosure, each of the dynamic stabilization rods has an integral or unitary construction formed from a single piece of material.

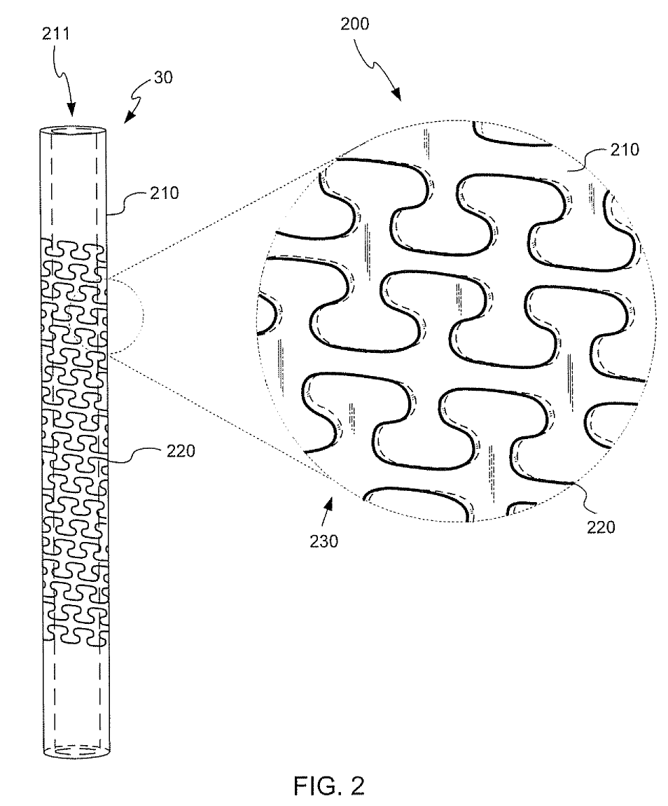

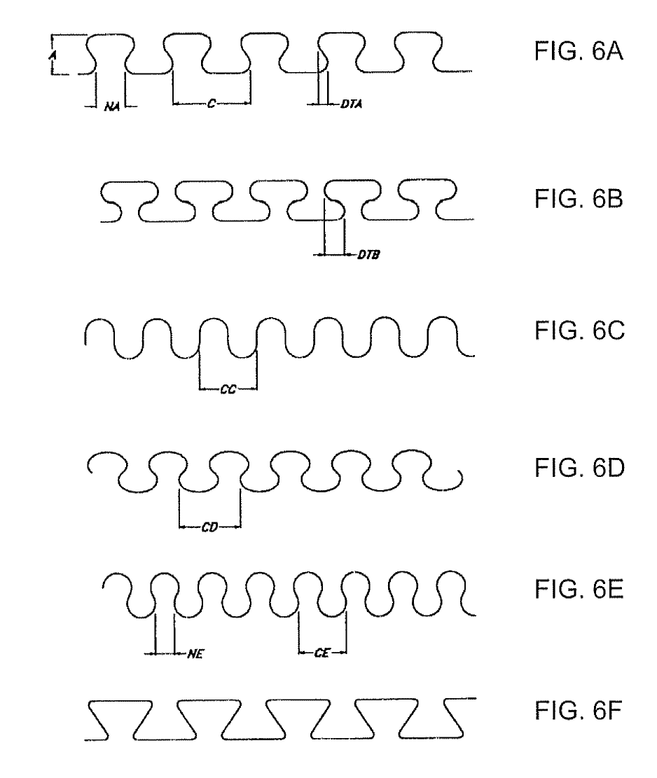

FIG. 2 depicts a schematic representation of a dynamic stabilization rod 30 for spinal implants and an exploded view 200 showing a detailed portion of the dynamic stabilization rod 30, according to one embodiment of the disclosure. In this example, the dynamic stabilization rod 30 has a cylindrical body 210 having a cannulated interior 211 and an opening 220 extending spirally around a longitudinal axis of the cylindrical body 210. In one embodiment, as illustrated in the exploded view 200, the opening 220 can form an interlocking pattern 230. In one embodiment, the interlocking pattern has a shape of or resembles a dog bone or puzzle. In one embodiment, the opening 220 has a non-linear path. As will be described later with reference to FIGS. 6A-6K and 11-12, other patterns are also possible, including those formed by one or more linear or non-linear paths.

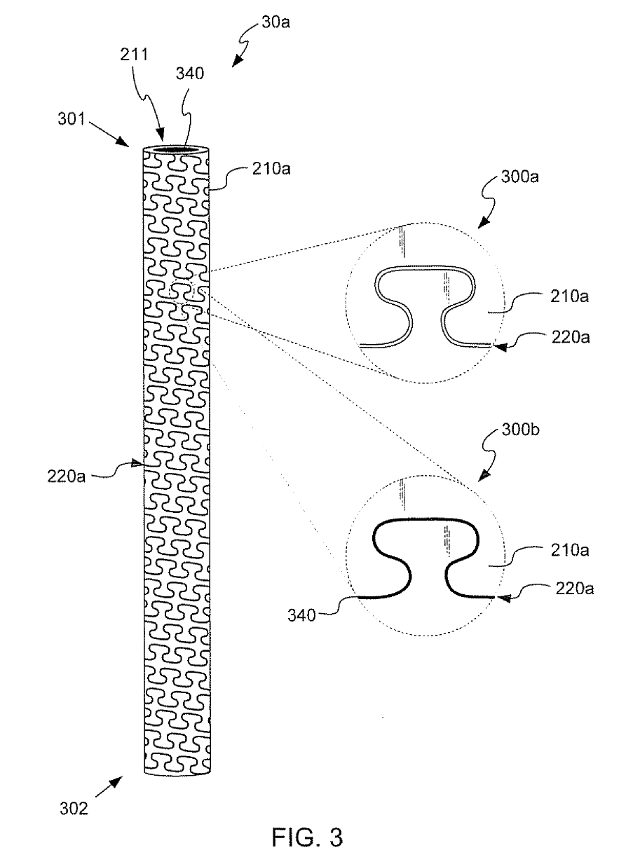

FIG. 3 depicts a schematic representation of a dynamic stabilization rod 30a and exploded views 300a and 300b showing detailed portions of the dynamic stabilization rod 30a, according to one embodiment of the disclosure. As FIG. 3 exemplifies, in one embodiment, the dynamic stabilization rod 30a may comprise a biomaterial 340 filling the interior 211 in whole or in part In one embodiment, the cylindrical body 210a may be coated in whole or in part with the biomaterial 340. In one embodiment, the biomaterial 340 at least partially fills the opening 220a. In the example of FIG. 3, the exploded view 300a shows a portion of the opening 220a unfilled and the exploded view 300b shows a portion of the opening 220a filled with the biomaterial 340.

In one embodiment, the biomaterial 340 is a polymer. In one embodiment, the biomaterial 340 is polycarbonate urethane. Other biomaterials are also possible.

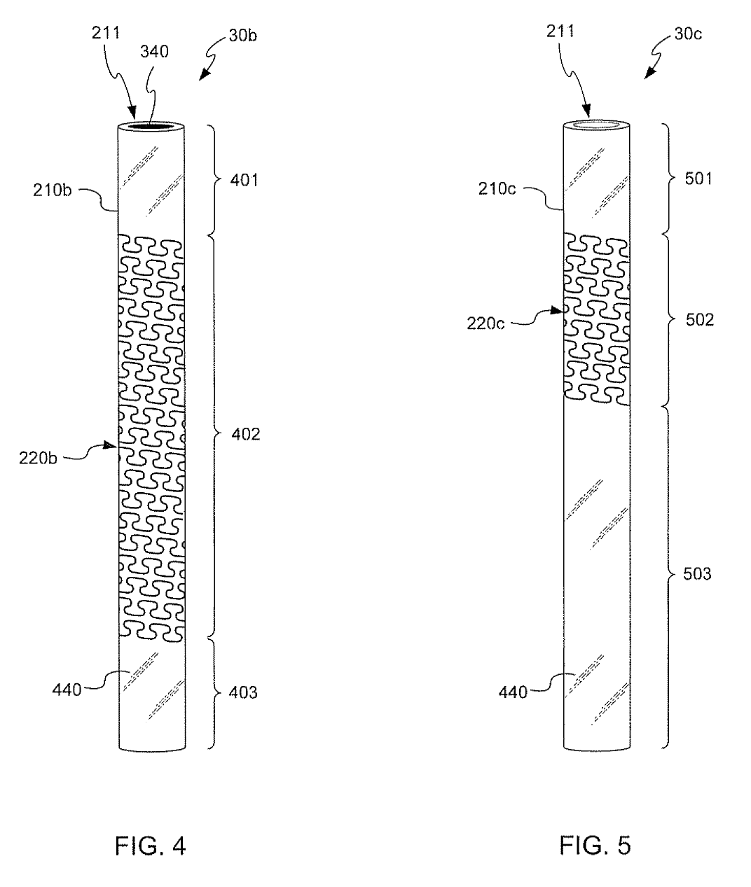

As an example, the opening 220a is shown in FIG. 3 to cover just about the entire cylindrical body 210a of the dynamic stabilization rod 30a, between a first end 301 and a second end 302. As FIGS. 4-5 exemplify, the opening(s) of a dynamic stabilization rod according to this disclosure can have various lengths and/or be positioned at various portion(s) of the cylindrical body. More specifically, FIG. 4 depicts a schematic representation of a dynamic stabilization rod 30b having a cylindrical body 210b and a cannulated interior 211. In this example, the dynamic stabilization rod 30b is filled with the biomaterial 340. In the configuration shown in FIG. 4, the cylindrical body 210b of the dynamic stabilization rod 30b has three portions or sections 401, 402, and 403 and the opening 220b extends longitudinally about the mid section 402. FIG. 5 depicts a schematic representation of a dynamic stabilization rod 30c having a cylindrical body 210c and a cannulated interior 211. In this example, the dynamic stabilization rod 30c is hollow inside. In the configuration shown in FIG. 5, the cylindrical body 210c of the dynamic stabilization rod 30c has three portions or sections 501, 502, and 503 and the opening 220c is asymmetrically positioned in one of the sections.

FIGS. 6A-6K depict schematic representations of various patterns for implementing embodiments of dynamic stabilization rods disclosed herein. The patterns are representative of patterns with non-linear paths which can be used and are not intended to be all inclusive. Other patterns are also possible. For example, a dynamic stabilization rod may embody a line that forms a linear path or lines that form a non-contiguous linear path spirally around the longitudinal axis of the cylindrical body.

The pattern illustrated in FIG, 6A has a cycle length C, which includes a neck region NA. The wider the neck region the greater the torsional forces which a dynamic stabilization rod can transmit. The ability of a dynamic stabilization rod to interlock is dependent in part upon the amount of overlap or dovetailing, indicated as DTA in FIG. 6A and DTB in FIG. 6B. The pattern of 6C, does not provide dovetailing, and requires a helix angle which is relatively small. FIG. 6D illustrates a segmented, elliptical dovetail configuration with CD indicating the cycle of repetition. In FIG. 6E, the ellipse has been rounded out to form a circular dovetail cut with CE indicating the repetitive cycle and the cut pattern of FIG. 6F is a dovetailed frustum. The pattern of FIG. 6G is a sine wave pattern forming a helical path. FIG. 6H is an interrupted (i.e., non-contiguous) spiral in which the opening follows a helical path, deviates from the original angle for a given distance, and then resumes the original or another helix angle. In this case, there are two lead cuts. FIG. 61 depicts a similar pattern as the one shown in FIG. 6H with a different pitch and a single lead cut. FIGS. 6J and 6K show two dimensions of the same pattern having multiple leads.

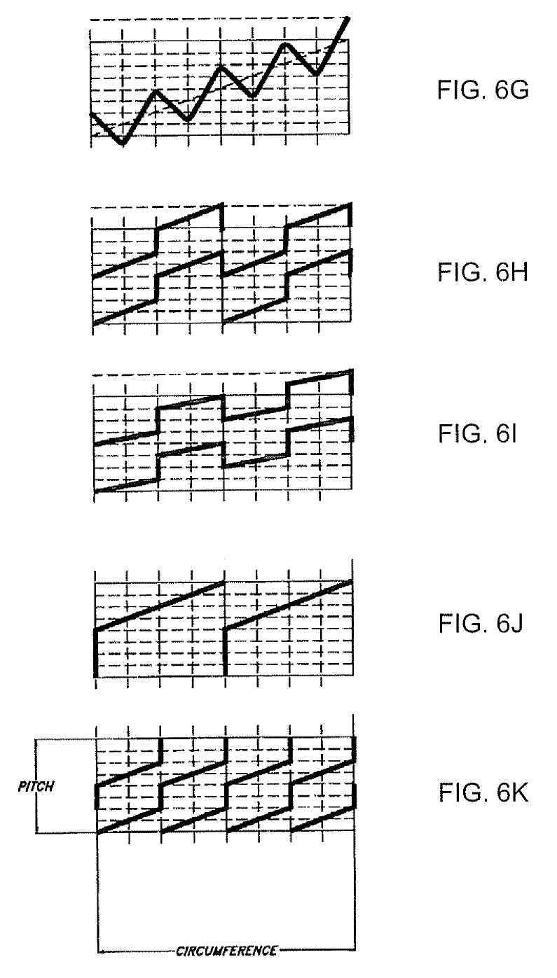

FIGS. 7A-7C depict schematic representations of side views each showing a portion of a spiral opening of a dynamic stabilization rod having a distinct pitch according to some embodiments of the disclosure. As shown in FIG. 7C, with certain patterns, a rotational force in the direction of arrow 710 may expand the spiral opening of a dynamic stabilization rod. The steeper angles of FIGS. 7B and 7C provide progressively greater resistance to opening. As illustrated in FIGS. 7A, 7B and 7C, a spiral opening without a dovetail may be configured with an odd number of cycles per revolution to provide an adequate structural strength. In this manner, the peak point 71 of a first revolution is out of phase with the peak point 72 of a second revolution. A steep helix angle (i.e., a very low number of cycles per revolution) can be used to provide adequate space between the peak points 71 and 72. Dovetailed interlocking patterns such as the dog pattern 230 shown in FIG. 2 can provide greater resistance to opening.

According to one feature of the disclosure, a dynamic stabilization rod can be produced by many convenient means. For example, computer-controlled machining techniques, including computer-controlled milling or cutting, wire electrical discharge machining, water jet machining, spark erosion machining, and laser cutting can be readily utilized to produce a dynamic stabilization rod with a desired pattern

The advantages of using a computer controlled laser cutting technique are the infinite variety of patterns which can be produced, the ability to change the helix angle at any point along the rod, the variations with respect to opening width, and the overall precision it provides. Laser cutting the patterns can produce customized dynamic stabilization rod having a predetermined flexibility with predetermined variations in the flexibility while providing substantially uniform characteristics with counterclockwise and clockwise rotation.

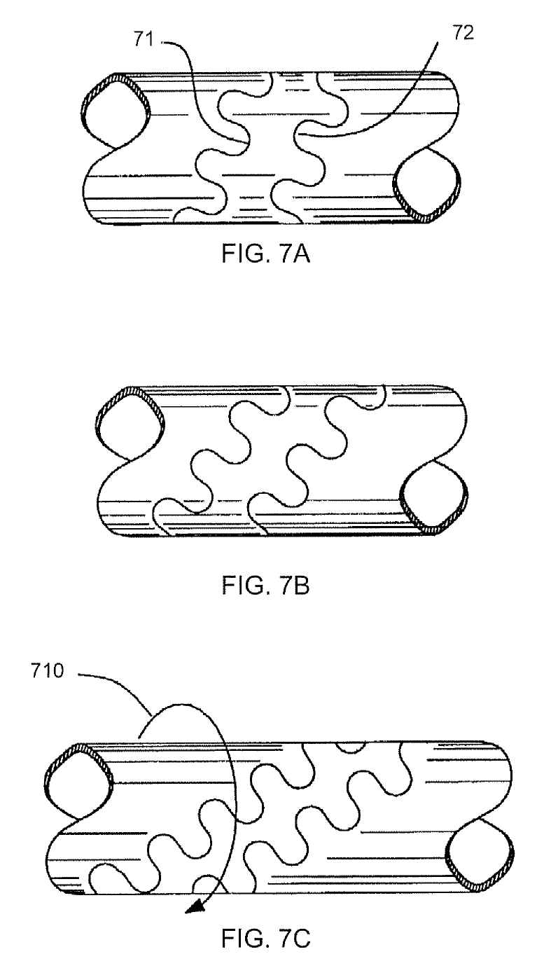

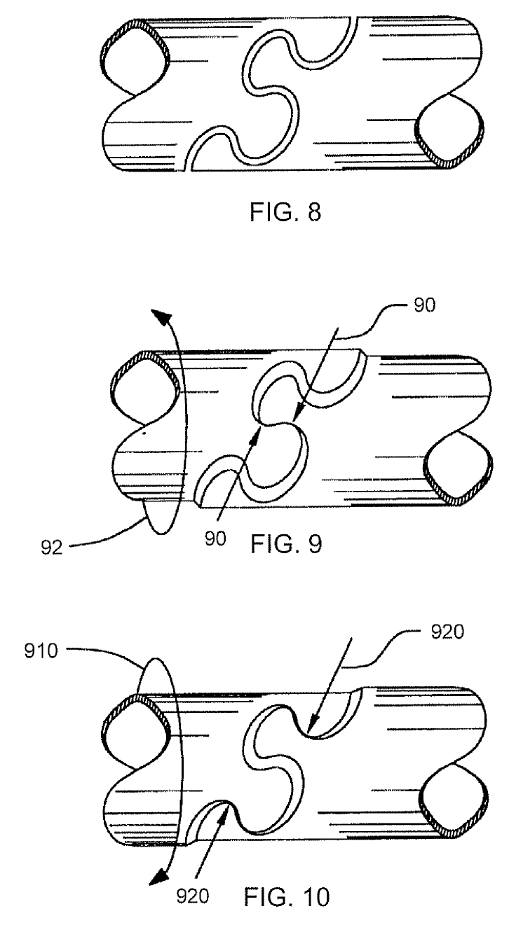

The effect of the rotational forces on a flexible dynamic stabilization rod is demonstrated in FIGS. 8, 9 and 10. FIG. 8 depicts a schematic representation of a side view of a portion of a spiral opening of a dynamic stabilization rod in a normal state. FIGS. 9-10 depict schematic representations of side views of the portion of the spiral opening of the dynamic stabilization rod of FIG. 8 under rotational forces. More specifically, as shown in FIG. 9, rotation in the direction of arrow 92 applies a force in the direction of arrow 92, at the neck region, making contact at point 90. Conversely, as shown in FIG. 10, rotation in the direction of arrow 910 applies a force in the direction of arrow 910 at the neck region, making contact at point 920.

As described above, according to the disclosure, a dynamic stabilization rod for spinal implants can be made with a desired pattern utilizing a variety of machining techniques. In one embodiment, a method of making a dynamic stabilization rod comprises the steps of forming a cylindrical body from a first biomaterial, removing (e.g., drilling) the first biomaterial from inside of the cylindrical body along a longitudinal axis of the cylindrical body to form a cannulated interior and machining an opening about the longitudinal axis of the cylindrical body. These steps may be performed in order or in no particular order. As described above, the opening may have one or more cut leads. The helical path or paths forming the opening may be linear or non-linear.

In one embodiment, the machining step may further comprise rotating the cylindrical body around the longitudinal axis, moving the cylindrical body in an axial direction, and following a predetermined non-linear path, continuously or intermittently cutting away the first biomaterial from the cylindrical body. The predetermined non-linear path may correspond to a recurring shape of a dog bone or puzzle, according to one embodiment of the disclosure. The dog bone pattern can facilitate compression and mitigate the rotational forces.

The above-described methods may include a step of coating the cylindrical body in whole or in part and/or a step of filing the cannulated interior in whole or in part with a suitable biomaterial. In one embodiment, the suitable biomaterial is a polymer. In one embodiment, the suitable biomaterial is polycarbonate urethane. According to one feature of the disclosure, coating the cylindrical body can improve wear resistance According to one feature of the disclosure, filing the cannulated interior in whole or in part with a suitable biomaterial can enhance the rigidity of the dynamic stabilization rod.

Unlike prior rods which are often wholly rigid, the dynamic stabilization rods disclosed herein are quite flexible and can permit some range of motion for a patient who has undergone spinal implant surgery. This flexibility can be controlled via a careful selection of the first biomaterial described above and/or a plurality of factors affecting the physical configuration of the opening (e.g., the pattern chosen to form the opening, the number of cycles per revolution, the pitch or angle of the pattern, the location of the opening, the length of the opening, the width of the opening, the outer and inner diameters of the cylindrical body, etc.). Depending upon the first biomaterial, the configuration of the opening, and/or the need(s) of a patient, it may be the case that some rigidity of the dynamic stabilization rod is desired. In one embodiment, the method of making a dynamic stabilization rod further includes a step of at least partially filing the opening with a suitable biomaterial (e.g., polycarbonate urethane). One advantage of at least partially filing the opening is that the rigidity of the dynamic stabilization rod can be further enhanced.

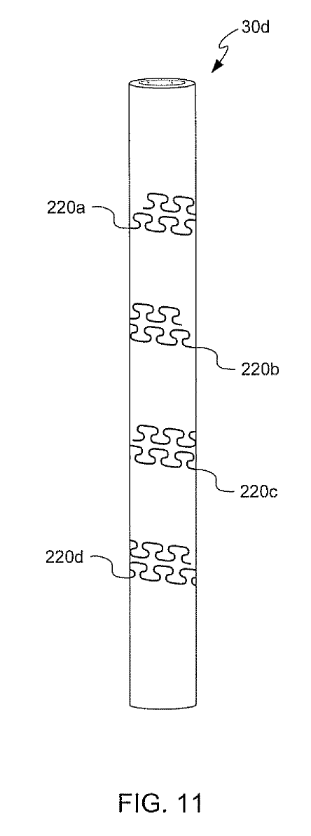

Another way to control the flexibility or flexing capability of a dynamic stabilization rod is to strategically segment the cylindrical body of the rod. As an example, FIG. 11 depicts a schematic representation of a dynamic stabilization rod 30d with more than one spiral opening (erg., 220a, 220b, 220c, and 220d), according to one embodiment of the disclosure.

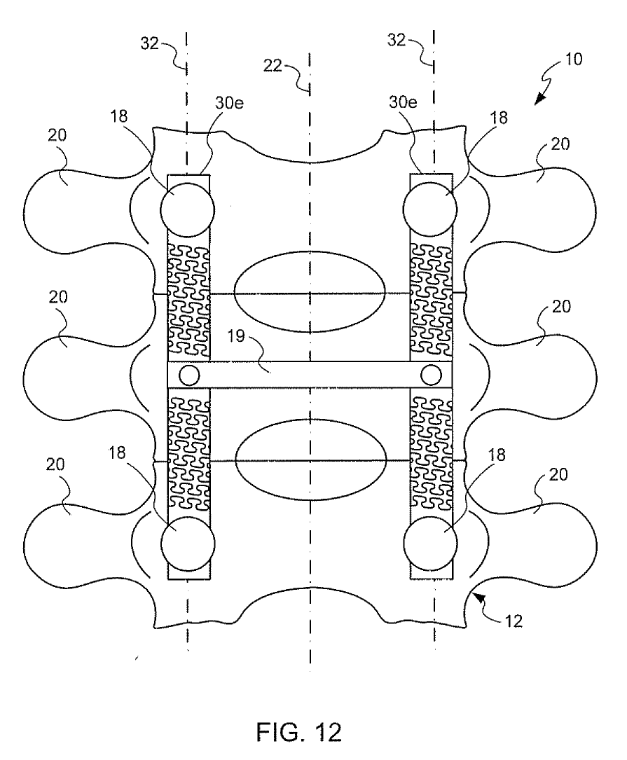

The additional rigidity provided by the strategic segmentation of the cylindrical body of a dynamic stabilization rod can be further utilized in other applications For example, FIG. 12 depicts a schematic representation of a top view showing a spinal implant system in use and including a pair of dynamic stabilization rods 30e. In this example, each dynamic stabilization rod 30e has more than one opening and an uncut portion of the cylindrical body is configured to receive a connection for another component of a spinal implant system 10 (e.g., a cross-link connection 19). As FIG. 12 exemplifies, portion(s) of the cylindrical body can be left rigid and uncut for integration with other spinal device(s) to facilitate fusion or segmental stability of the spine According to one embodiment of the disclosure, the cylindrical body has a uniform cylindrical shape that is compatible with a variety of anchoring systems 18 and/or connections 19 Other configurations are possible, such as, for example, solid prismatic shaped rod portions or elliptical shape or helical shape

In any of the previously described embodiments, an elongate hole may extend through the entire length of the dynamic stabilization rod, centered on the axis 32, such as shown in FIG. 2, as yet another means of achieving the desired initial bending stiffness/bending moment of inertia. In this regard, the diameter of the cannulated interior 211 could be enlarged to provide a lower bending stiffness/bending moment of inertia around the opening 220. It should also be appreciated that, as with conventional non-dynamic rods, the dynamic stabilization rods 30 can be permanently deformed or bent to match a desired curvature of the corresponding portion of the spine 12 and that this permanent deformation can either be preformed by the manufacturer or custom formed by the surgeon during a surgical procedure.

The system 10 according to the disclosure may be used in minimally invasive surgery (MIS) procedures or in non-MIS procedures, as desired, and as persons of ordinary skill in the art who have the benefit of the description of the disclosure understand. MIS procedures seek to reduce cutting, bleeding, and tissue damage or disturbance associated with implanting a spinal implant in a patient's body Exemplary procedures may use a percutaneous technique for implanting longitudinal rods and coupling elements. Examples of MIS procedures and related apparatus are provided in U.S. patent application Ser. No. 10/698,049, filed Oct. 30, 2003, U.S. patent application Ser. No. 10/698,010, filed Oct. 30, 2003, and U.S. patent application Ser. No. 10/697,793, filed Oct. 30, 2003, incorporated herein by reference. It is believed that the ability to implant the system 10 using MIS procedures provides a distinct advantage.

Persons skilled in the art may make various changes in the shape, size, number, and/or arrangement of parts without departing from the scope of the disclosure as described herein. In this regard, it should also be appreciated that components of the system 10 shown in the figures are for purposes of illustration only and may be changed as required to render the system 10 suitable for its intended purpose,

In the foregoing specification, the disclosure has been described with reference to specific embodiments. However, as one skilled in the art can appreciate, embodiments of the dynamic stabilization rod disclosed herein can be modified or otherwise implemented in many ways without departing from the spirit and scope of the disclosure. Accordingly, this description is to be construed as illustrative only and is for the purpose of teaching those skilled in the art the manner of making and using embodiments of a dynamic stabilization rod. It is to be understood that the forms of the disclosure herein shown and described are to be taken as exemplary embodiments. Equivalent elements or materials may be substituted for those illustrated and described herein. Moreover, certain features of the disclosure may be utilized independently of the use of other features, all as would be apparent to one skilled in the art after having the benefit of this description of the disclosure.

1. A dynamic stabilization rod for spinal implants, comprising:
a cylindrical body having a cannulated interior and an opening extending spirally around a longitudinal axis of the cylindrical body; and
a biomaterial coating the cylindrical body in whole or in part and at least partially filling the opening.

2. The dynamic stabilization rod of claim 1, wherein the opening has an interlocking pattern.

3. The dynamic stabilization rod of claim 2, wherein the interlocking pattern has a shape of a dog bone or puzzle.

4. The dynamic stabilization rod of claim 1, wherein the opening has a non-linear path.

5. The dynamic stabilization rod of claim 1, wherein the opening has a linear path.

6. The dynamic stabilization rod of claim 1, wherein the cylindrical body has a mid section and wherein the opening extends longitudinally about the mid section.

7. The dynamic stabilization rod of claim 1, wherein the cylindrical body has a first end and a second end and wherein the opening is positioned between the first end to the second end.

8. The dynamic stabilization rod of claim 1, wherein the biomaterial fills the cylindrical body in whole or in part.

9. The dynamic stabilization rod of claim 1, wherein the biomaterial is polycarbonate urethane

10. A spinal stabilization system, comprising:
a set of bone fasteners for anchoring the spinal stabilization system onto vertebral bodies; and
a dynamic stabilization rod connecting the set of bone fasteners, wherein the dynamic stabilization rod comprises a cylindrical body having a cannulated interior and an opening having a non-linear path and extending spirally around a longitudinal axis of the cylindrical body.

11. The spinal stabilization system of claim 10, wherein the non-linear path forms an interlocking pattern.

12. The spinal stabilization system of claim 11, wherein the interlocking pattern has a shape of a dog bone or puzzle.

13. The spinal stabilization system of claim 10, wherein the dynamic stabilization rod further comprises a polycarbonate urethane biomaterial coating the cylindrical body in whole or in part and at least partially filling the opening.

14. The spinal stabilization system of claim 13, wherein the polycarbonate urethane biomaterial filling the cylindrical body in whole or in part.

15. A method of making a dynamic stabilization rod for spinal implants, comprising:
forming a cylindrical body from a first biomaterial;
removing the first biomaterial from inside of the cylindrical body along a longitudinal axis of the cylindrical body to form a cannulated interior;
machining an opening circumferentially around at least a portion of the cylindrical body; and
at least partially filing the opening with a second biomaterial to enhance rigidity of the dynamic stabilization rod, wherein the second biomaterial is a polymer.

16. The method of claim 151 wherein the second biomaterial is polycarbonate urethane.

17. The method of claim 15, further comprising:
coating the cylindrical body in whole or in part.

18. The method of claim 17, further comprising:
filing the cannulated interior in whole or in part with the second biomaterial.

19. The method of claim 15, further comprising:
filing the cannulated interior in whole or in part with the second biomaterial.

20. The method of claim 15, wherein the machining further comprises:
rotating the cylindrical body around the longitudinal axis;
moving the cylindrical body in an axial direction; and
following a predetermined non-linear path, continuously or intermittently cutting away the first biomaterial from the cylindrical body, wherein the predetermined non-linear path corresponds to a recurring shape of a dog bone or puzzle.

21. The dynamic stabilization rod made according to the method of claim 20.

22. The method of claim 15, wherein the steps are performed in the order of:
1) forming the cylindrical body from the first biomaterial;
2) removing the first biomaterial from inside of the cylindrical body along the longitudinal axis of the cylindrical body to form the cannulated interior;
3) machining the opening about the longitudinal axis of the cylindrical body; and
4) at least partially filing the opening with the second biomaterial

23. The dynamic stabilization rod made according to the method of claim 22.

24. The dynamic stabilization rod made according to the method of claim 15.

25. A method of making a dynamic stabilization rod for spinal implants, comprising:
forming a cylindrical body from a first biomaterial;
removing the first biomaterial from inside of the cylindrical body along a longitudinal axis of the cylindrical body to form a cannulated interior;
machining an opening about the longitudinal axis of the cylindrical body;
at least partially filing the opening with a second biomaterial to enhance rigidity of the dynamic stabilization rod, wherein the second biomaterial is a polymer; and
coating the cylindrical body in whole or in part with the polymer.

26. The method of claim 25, wherein the second biomaterial is polycarbonate urethane

27. The method of claim 25, further comprising:
filing the cannulated interior in whole or in part with the second biomaterial.

28. The method of claim 25, wherein the machining further comprises:
rotating the cylindrical body around the longitudinal axis;
moving the cylindrical body in an axial direction; and
following a predetermined non-linear path, continuously or intermittently cutting away the first biomaterial from the cylindrical body, wherein the predetermined non-linear path corresponds to a recurring shape of a dog bone or puzzle.

29. The dynamic stabilization rod made according to the method of claim 28.

30. The method of claim 25, wherein the steps are performed in the order of:
1) forming the cylindrical body from the first biomaterial;
2) removing the first biomaterial from inside of the cylindrical body along the longitudinal axis of the cylindrical body to form the cannulated interior;
3) machining the opening about the longitudinal axis of the cylindrical body; and
4) at least partially filing the opening with the second biomaterial.

31. The dynamic stabilization rod made according to the method of claim 30.

32. The dynamic stabilization rod made according to the method of claim 25.
